(19) United States
(12) Patent Application Publication
(10) Pub. No.: US 2017/0265787 A1
(43) Pub. Date: Sep. 21, 2017

(54) Superfast Sequential and Alternate Dual Wavelength Reflection Technique

(71) Applicant: Airware, Inc., Goleta, CA (US)

(72) Inventor: Jacob Y Wong, Goleta, CA (US)

(73) Assignee: Airware, Inc., Goleta, CA (US)

(21) Appl. No.: 15/075,559

(22) Filed: Mar. 21, 2016

(51) Int. Cl. A61B 5/1455; A61B 5/00; A61B 5/145

(52) U.S. Cl. CPC A61B 5/1455; A61B 5/14532; A61B 5/0022; A61B 5/681

(57) ABSTRACT

Two coherent narrow bandwidth infrared beams, the Signal and the Reference, are incident at an angle and at high frequency sequentially and alternately at the same spot of a whole blood/body tissues sample. The Signal beam has a center wavelength which falls within an absorption line of glucose in whole blood (e.g. 1.409μ). The Reference beam has a center wavelength which does not coincide with any known absorption lines of glucose in whole blood (e.g. 1,278μ). Radiation emitted from the spot at which the beams penetrate into the sample and subsequently emanate from it after multiple scattering and spurious absorption effects is collected by a lens onto an infrared detector. The ratio of the voltage detected from the emerging Signal beam over that of the Reference beam is processed to yield the value of glucose concentration in the whole blood/body tissue sample.

### FIELD OF THE INVENTION

This invention relates to medical instrumentation, and more specifically, to the field of measurement of blood glucose concentration levels.

### BACKGROUND OF THE INVENTION

Diabetes is a disease in which the body's natural control of blood sugar (glucose) has been lost. Diabetic patients must frequently measure blood glucose in order to be able to maintain their health and lead relatively normal lives. At present there are two general methods by which glucose control can be quantified over time: self blood glucose monitoring by individual subject and measurement of glycosylated proteins. Both of these approaches require a sample of blood. Home blood glucose monitoring provides information regarding the blood glucose at the moment of measurement and allows immediate correction of metabolic problems. Measurement of glycosylated proteins allows quantification of glucose control over a longer period of time such that the overall efficacy of management strategy can be determined. Current glucose analytical systems require blood to be extracted from the body prior to performing the analysis. This blood withdrawal requirement limits the application of such testing; many people who may be interested in knowing their glucose levels are reluctant to have either their finger poked or blood samples removed by hypodermic needle.

Over the past several decades, literally hundreds of noninvasive blood glucose measurement techniques ranging from spectroscopic absorption, transmission, reflectance and scattering utilizing near-, mid- and far-infrared radiation, photoacoustic resonance, optical polarization, light scattering, Raman scattering, transdermal analysis and dozens of other sophisticated techniques have been suggested and tried without success. An excellent summary of these failed techniques is set forth in the book entitled “The Pursuit of Noninvasive Glucose” (Fourth Edition 2015) by John L. Smith. Among the many measurement techniques attempted, the use of glucose absorption bands in the mid- and far-infrared regions appeared to be the most promising in earlier years. However, the measurement of physiological concentrations of glucose in blood by conventional infrared absorption spectroscopy has been severely hampered by the weak absorption of glucose and extremely high background absorption (^{˜}100-1,000 cm⁻¹) of liquid water in the infrared spectral region above ^{˜}1.5μ (6,700 cm−1). Based upon the numerous failures experienced and reported by many researchers in recent years, the use of infrared absorption techniques for measuring noninvasively glucose concentration levels in blood has largely been ignored or determined to be unsuccessful.

### SUMMARY OF THE INVENTION

The present invention is generally directed to an apparatus and method which non-invasively measure a glucose concentration in a human subject by alternating pulsing of narrow bandwidth coherent signal and reference beams at a fast cycle speed (e.g., 10 Khz or more) toward a body surface at an incident angle such that the pulsed beams enter into an in-vivo sample area of the subject's body and are then collected onto a detector while the ratio of the emerging signal beam/emerging reference beam measured at the detector is used to determine the glucose concentration of the subject. The signal beam has an absorption peak at CWL=λ_{S} wherein λ_{S} is an absorption line of glucose which preferably has a liquid water attenuation no greater than ^{˜}1.0 cm⁻¹ (e.g., 1.409μ, 1.28μor 0.960μ with FWHM=+/−0.0054μ) while the reference beam has an absorption peak CWL=λ_{R} wherein λ_{R} has no or negligible absorption by glucose molecules (e.g., 1.28μ with FWHM=+/−0.0054μ) and the wavelengths of the signal and reference beams are sufficiently close so that attenuation of both by virtue of scattering outside of the incident direction is substantially the same. The emerging signal and reference beams are collected by a focusing lens which has a collecting angle less than the incident angle to avoid collecting portions of the signal and reference beams that are reflected from the subject's body and do not enter the in-vivo sample area. Although the apparatus and method can be adapted for use with many different parts of a subject's body, it is especially preferred that they be used with a monitor that can be worn as, or combined with, a wristwatch, which can then communicate with a computer, such as a smart phone, for ease of monitoring, recordation and display of glucose concentration measurements, not only instantaneously, but also over a preselected period of time.

Accordingly, it is a primary object of the present invention to satisfy a long-felt need for a non-invasive blood glucose monitor.

This and further objects and advantages will be apparent to those skilled in the art in connection with the drawings and detailed description of the invention set forth below.

### BRIEF DESCRIPTIONS OF THE DRAWINGS

FIG. 1 depicts the absorbance of glucose in water for different concentrations.

FIG. 2 depicts the design of an optical system according to the present invention for measuring the absorbance of different glucose concentrations while eliminating the absorptive scattering effects of constituent molecules in human blood.

FIG. 3 depicts one of the best sampling areas for monitoring non-invasively the glucose concentration in a human's blood/interstitial tissues.

FIG. 4 depicts the design detail of a watch-like non-invasive glucose monitor utilizing the infrared double-beam reflection technique of the present invention.

### DETAILED DESCRIPTION OF THE INVENTION

Glucose in human blood exists as an independent and freely-standing molecule even though it is also attached to almost all proteins of the body. As a matter of fact, it is this fondness for proteins that causes many of the complications of diabetes when the level of freely-standing blood glucose is not well controlled. The glucose molecule is known to be stable or indissociable to excitation by absorption of specific wavelengths of light via its absorption bands. Identification of absorption bands for freely-standing glucose molecules in the near-infrared (NIR) spectral region (^{˜}0.6-1.5 μ) in blood is important because of the relatively low background absorption of water (absorption coefficient <10 cm⁻¹) in this spectral region versus extremely high background absorption of water (>1,000 cm⁻¹) in the mid- to far-infrared spectral regions.

Physiologically speaking, glucose exists in blood or body tissues in two forms. The first form is simply a standalone or freely-standing molecule. The second form is when it attaches to some other constituent molecules in blood such as all kinds of proteins commonly known as “glycosylated” proteins after glucose attachment. But it is only the concentration of freely-standing glucose molecules in blood or in body tissues that is of medical importance and interest. From the scientific standpoint, this form of glucose is also of great importance because absorption bands of freely-standing glucose molecules, once identified, can be uniquely assigned and searched. Furthermore, their absorption characteristics (e.g. transmittance to radiation) should stay invariant or unchanged as long as they find themselves in a non-interacting environment.

However, the absorption bands of freely-standing glucose molecules in blood and/or in body tissues are not free from interferences affecting them, not only by water molecules which provide an attenuating background shield against incoming radiation of specific wavelengths, but also by other constituent molecules having comparable concentrations as them in blood and/or in body tissues. These constituent molecules include “glycosylated” proteins, serum proteins, blood cells, hemoglobin, fatty acids, lipids, minerals, hormones and other waste products. They interfere with glucose molecules via collisions, absorbing radiation destined for glucose and/or scattering the incoming radiation away from the targeted detector thus affecting the absorption characteristics of glucose molecules and their absorption bands. These interferences will now be described in more detail below.

There are three interference effects one has to heed that might alter the measured absorptive characteristics of glucose absorption lines. The first interference effect is spectral broadening of glucose absorption lines due to collisions of glucose with other constituent molecules. If this occurs, peak absorption strength of the lines will likely be reduced because of widening of the absorption line shape. The second possible interference effect is partial spectral overlapping of glucose absorption lines by absorption lines of other constituent molecules. When this occurs, the measured absorption characteristics, like transmittance to incoming radiation, will not only be a function of the glucose concentration level, but also the concentration level of those molecules whose absorption line or lines partially overlap that of glucose. The third interference effect is scattering of radiation originated from the source towards the detector by all molecules, including those of glucose and liquid water, in the sampling matrix. Such scattering not only can alter in a significant way the magnitude of the absorption signal assigned to the measurement of glucose molecules, it can also generate undesirable noise sources for the measurement.

One of the principal reasons why so many techniques covering just about all the scientific disciplines available to researchers over the past several decades failed to realize a non-invasive blood glucose sensor can be attributed to one amazingly simple fact and that is the failure to establish a reliable and time-invariant reference for the measurement. This is no doubt because glucose molecules (whose concentration one would like to measure, non-invasively or otherwise) exist in a very complex medium or matrix inside the human body comprising a large multitude of all kinds of constituent molecules. These molecules include blood cells, hemoglobin, serum and glycosylated proteins, lipid, fatty acids, melanin, collagen, hormones, minerals and other waste products. Moreover, all these constituent molecules, as well as glucose molecules themselves, are in constant motion over time, albeit very slowly (only at a few mm/sec). At any one instant when a blood glucose concentration measurement is made, there is no way to identify the configuration and composition of these constituent molecules surrounding the glucose molecules. In other words, a reliable and time-invariant reference for any measurement irrespective of its scientific technique simply cannot be found. Without a reliable and time-invariant reference, the calibration of the measurement will always be questionable and inaccurate leading indirectly to the failure of the measurement technique itself. This is precisely the underlying real problem just about every past researcher failed to recognize and solve in the pursuit of realizing a non-invasive blood glucose monitor.

An example of this problem can be illustrated by a method of glucose concentration measurement using direct infrared absorption by glucose molecules in blood. Glucose molecules have several weak absorption bands in the near infrared spectral region (1.0-1.5μ). Assume now that we will use one of these absorption lines, e.g. 1.409μ, for the glucose measurement. We will use in this example the distance between the infrared source and the infrared detector as the effective path length of the sample chamber. An infrared photon emitted from the infrared source on its way to the infrared detector can have more than one fate. The first one is the case where there are no glucose molecules in between the source and the detector. The photon will arrive safely at the detector generating an output that represents the absence of glucose in the sample chamber. The second case is the situation where the photon does not show up at all at the detector. In this situation there can be two possibilities. The first one is that the photon is being absorbed by a glucose molecule. The assumption in this example here is that there are no molecules other than those of glucose that can absorb this photon of a very specific wavelength. The second situation is that even though there are glucose molecules in the sample chamber, the photon is not being absorbed by them. It is rather being scattered outside of the line of sight to the detector by other constituent molecules surrounding those of glucose. As a result it misses the detector and is not being detected.

As stated earlier, glucose molecules whose concentration one would like to measure exist in a very complex medium or matrix inside the human body comprising a large number of diverse constituent molecules. Radiation coinciding with one absorption line of a glucose molecule traversing in this complex medium can either be absorbed or be scattered out of the line of sight to the detector by constituent molecules surrounding those of glucose. The common practice is to use the amount of radiation absorbed by glucose molecules as reflected in the detector output as a measure of their concentration in the medium. This practice is valid when the matrix through which the measuring radiation passes is relatively thin or dilute like the atmosphere where air molecules are located far apart. In such a thin matrix, scattering loss by the measuring radiation is rare and the detector output will correctly indicate the amount of radiation loss due to absorption by the relevant molecules in the matrix.

Such is not the case when the matrix is very complex and ‘dense’ like the glucose molecules in the medium of blood/interstitial tissues. The measuring radiation which can be absorbed by glucose molecules in the matrix of blood/interstitial tissues on its way to the detector can also be scattered by constituent molecules surrounding those of glucose and therefore not reach the detector for detection. Furthermore, since the constituent molecules surrounding the glucose molecules in the blood/interstitial tissues matrix are in constant motion, albeit relatively slowly, the radiation loss at the detector due to scattering will vary as a function of time. Thus it will be difficult or even impossible to determine glucose concentration reliably based on the detector signal level when the matrix is complex like that of the blood/interstitial tissue.

The example of detecting glucose concentration in blood/interstitial tissues discussed above clearly points out the fact that the problem does not lie in the measurement technique like using glucose infrared absorption—rather, it is caused by the time-variant complex medium surrounding the glucose molecules. Such a matrix simply will not allow establishment of a reliable and time-invariant reference for the measurement. When the reference of the measurement changes unpredictably over time, the accuracy and reliability of the measurement cannot be sustained. Failure to recognize and solve this underlying real problem for measuring glucose concentration in blood/interstitial tissues, namely the ability to identify a time-invariant reference for the matrix and not the measurement technique, has prevented a non-invasive blood glucose sensor from being a reality for decades.

Recognizing the fact that the underlying real problem lies in the complex medium surrounding the glucose molecules for their concentration measurement, the present invention is directed to a special signal processing technique and apparatus that adequately addresses this problem and is thereby able to determine non-invasively human blood glucose concentration levels. The invention utilizes a special dual wavelength superfast pulsation technique directing two coherent narrow bandwidth laser beams at one and the same spot with their incident direction subtending an angle θᵢ from the surface normal at the impinging site. This technique uses two diode lasers of different wavelengths as the two radiation sources. One of the two diode lasers is selected to emit at a wavelength λ_{S} that has been experimentally verified to be absorbed by glucose molecules in rabbit blood with measured absorbance proportional to glucose concentration levels. The beam of this laser diode, Φ_{S}, with output radiation λ_{S}, will be designated as the Signal beam of the current invention apparatus. The second diode laser beam, Φ_{R}, is selected to emit at a wavelength λ_{R} which has been experimentally verified to have no or negligible absorption by glucose in rabbit blood. The beam of this laser diode, Φ_{R}, with output radiation λ_{R}, will be designated as the Reference beam of the current invention apparatus. In the selection process of λ_{S} and λ_{R}, respectively for the wavelengths of the Signal and Reference beams, special care is taken to ensure that there are only very low or negligible absorption at these wavelengths by the constituent molecules surrounding those of glucose in the sampling matrix of whole blood and/or interstitial fluid.

Both the Signal beam and the Reference beam laser diodes have to be sequentially and alternately pulsed at a very high speed, e.g. 10-100 KHz, each with a ^{˜}10% duty factor. Furthermore, their outputs are combined in a multiplex fashion via a fiber optical or beam-splitter means such that both beams traverse sequentially and alternately at different times the same short distance in and out of the matrix medium after impinging at the incident spot.

With the establishment of the Signal and the Reference beams Φ_{S} and Φ_{R}, respectively having radiation outputs of wavelength λ_{S} and λ_{R}, the current invention apparatus, in addition to the two laser diodes, is equipped with a quasi-sample chamber for accommodating the sampling matrix of whole blood and/or interstitial fluid. Both the Signal beam Φ_{S} and the Reference beams Φ_{R} are multiplexed to be directed at an angle θᵢ from the surface normal of the impinging spot. Both beams traverse the same distance sequentially and alternately at different times in and out of the medium matrix at the impinging spot. At the same time both beams are quasi specularly reflected from the impinging spot at an angle θᵣ=θᵢ from the surface normal. The reflected beams will not be used in the signal processing scheme of the current invention.

Both the Signal and the Reference beams that emerge from the impinging spot after traversing a short distance into the matrix medium are collected by a focusing lens onto an infrared detector. The collection angle θ_{c} as measured from the surface normal at the impinging spot is designed using a focusing lens with a collecting angle <θᵢ so that none of the incident Signal or Reference beams will be directed to the detector. The Signal emerging beam and the Reference emerging beam from the impinging spot are respectively labeled as Φ_{ES} and Φ_{ER}. As seen from the detector, the strength of Φ_{ES} and Φ_{ER} are almost the same except for the fact that if there are glucose molecules in the matrix medium at the impinging spot, then the strength of Φ_{ES} is less than that of Φ_{ER} due to absorption of Φ_{ES} by glucose molecules present in the matrix medium. Thus, by measuring the value of the ratio of Φ_{ES}/Φ_{ER} at the detector, the concentration of glucose in the matrix medium can be determined.

It is important to note that although the constituent molecules in the matrix of blood/body tissues including glucose are in constant motion, unlike air molecules in the atmosphere, they move about very slowly like several mm/sec instead of several km/sec. The fact that the time separation between the incident beams of Φ_{S} and Φ_{R} traveling through the same space in and out of the matrix medium sequentially and alternately is only a few μsec, the motion of the constituent molecules surrounding those of glucose can be considered as effectively stationary. In other words, the value of Φ_{ER} represents almost a time-invariant reference to Φ_{ES} which measures the concentration of glucose in the matrix medium. Thus the current invention addresses the underlying real problem for the measurement of glucose concentration in a medium matrix of blood/body tissues by eliminating the influence of the constituent molecules in the matrix in obtaining a reliable and time-invariant reference for the measurement.

The current invention first selects a glucose absorption line to work which has an absorption peak at CWL=λ_{S}. The liquid water attenuation at this selected glucose absorption line should ideally be no greater than ^{˜}1.0 cm⁻¹. Three absorption lines of glucose in the NIR (^{˜}0.9-1.5 μ) spectral region have been verified by direct infrared experiments to be viable with the present invention. Respectively, these three absorption lines are located at 0.960μ (10,400 cm⁻¹), 1.150μ (8,693 cm⁻¹) and 1.409μ (7,100 cm⁻¹) and they all have comparable glucose absorption strength. For the present invention, λ_{S} is selected to be 1.409μ (7,100 cm⁻¹) which is the strongest of the three. Needless to say, the other molecules present with glucose in the sampling matrix of whole blood and/or interstitial fluid might also have some small absorption at this spectral location of λ_{S}=1,409μ. It will be impossible to distinguish this absorption from that by glucose molecules. Any undesirable absorption other than that by glucose molecules, if present, can only be treated as noise in the processed signal of the currently invented apparatus (see below).

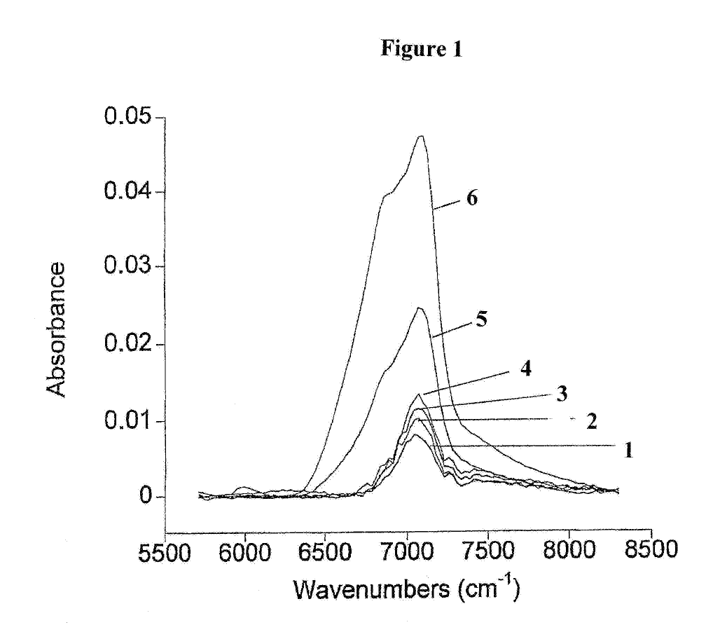

FIG. 1 shows the 1.409μ (7,100 cm⁻¹) absorption line of glucose in pure water (glucose solution) for six concentration levels 1 through 6. Level 1 denotes a concentration of 0.1 g/dL or 100 mg/dL, Level 2 denotes 0.5 g/DL, Level 3 denotes 1.0 g/dL, Level 4 denotes 2.0 g/DL, Level 5 denotes 5 g/dL and Level 6 denotes 10 g/DL. The symbol ‘dL’ denotes deci-Liter or 100 c.c. in the above labeling. The path length used in the measurement is 10 mm. One can see from FIG. 1 that the absorbance for 0.1 g/dL or 100 mg/dL of glucose in glucose solution (see 1 in FIG. 1) is ^{˜}0.003 which for a path length of 10 mm is equivalent to a signal attenuation of 0.7% of the incident radiation.

The current invention selects next a spectral location λ_{R} where not only there is no or negligible absorption by glucose molecules, there is also minimum absorption by all other molecules present with glucose in the sampling matrix of whole blood and/or interstitial fluid such as blood cells, hemoglobin (oxy- and deoxy-), serum proteins (albumin, gobulin, fibrinogen), glycosylated proteins, lipids, fatty acids, melanin, collagen, hormones, minerals and other waste products. The spectral location of λ_{R} selected for the current invention is 1.298μ (7,704 cm⁻¹). Any small absorption of this λ_{R} radiation by glucose molecules or by any other molecules present with them in the sampling matrix of whole blood and/or interstitial fluid will alter the value of V_{R} acting as a reference. As a reference, its presence will be considered as noise in the processed signal of the presently invented apparatus (see below).

The most important interference effect mentioned above is the scattering of the detector-directed incident radiation by the presence of all molecules, including glucose and liquid water, in the sampling matrix of whole blood and/or interstitial fluid. When NIR radiation is scattered by molecules in body tissue, the collisions are mostly elastic meaning that no energy is lost and the scattered photon merely changes direction. But a change in direction of photons having λ_{S} or λ_{R} missing the detector by virtue of scattering is equivalent to the photon being absorbed or a generation of noise in the signal processing system. Therefore, unless properly taken care of, scattering by all the molecules in the sampling matrix can alter in a significant way the magnitude of the absorption assigned to the measurement of glucose molecules.

In order to deal with this serious scattering problem, a special signal processing scheme comprising two radiation beams is setup in the current invention. The first beam Φ_{S} represents the Signal beam and comprises a pulsed semiconductor InGaAsP laser beam emitting at center wavelength (CWL)=1.409μ (7,100 cm⁻¹) with Full width at half maximum (“FWHM”)=+/−0.005μ. The second beam Φ_{R} represents the Reference beam and comprises a pulsed semiconductor InGaAsP laser beam emitting at center wavelength (CWL)=1.298μ (7,704 cm⁻¹) with FWHM=+/−0.005μ. The selection of the wavelengths for Φ_{S} and Φ_{R} takes advantage of the fact that for elastic scattering of radiation in liquid solution, like in the case that is currently being considered, the scattered angle is pretty much independent of the radiation wavelength as long as they are about the same. In other words, the attenuation of a beam by virtue of scattering outside of a targeted direction towards a detector is substantially the same for both Φ_{S} and Φ_{R} since λ_{S}≈λ_{R}. Thus, if we process the glucose absorption signal as a ratio S=V_{S}/V_{R}, the scattering effect on the measured signal S will tend to cancel each other and thereby significantly be reduced or eliminated. However, this is only true if the medium matrix is invariant in time for both Φ_{S} and Φ_{R}. But as we have mentioned earlier, the medium matrix actually is in constant slow motion and is therefore not time-invariant, unless some measuring scheme is devised to compensate for this fact, the scattering effects on the measured signal, namely S=V_{S}/V_{R}, will not be reduced or eliminated.

If however an almost identical sampling element of the matrix medium can be presented to the two beams in sequence during their respective measurements at slightly different times, the subsequent cancellation of the scattering effects for the two beams using that measurement scheme can be substantially reduced, if not eliminated. This situation is achieved by directing both the Φ_{S} and Φ_{R} beams sequentially and alternately at a very fast rate (e.g. 10-100 KHz) to traverse the same physical space through the matrix medium. This can be achieved by an optical technique using a fiber optical multiplexer or a special optical coating beam splitter (see below). Since the molecules in solutions or fluids like blood or interstitial tissues move relatively slowly (typically of the order of a few mm/sec), the time interval between the successive measurements by Φ_{S} and Φ_{R} is of the order of microseconds. Under this situation, the physical space encountered successively by Φ_{S} and Φ_{R} can be considered as stationary and identical. Since only Φ_{S} is absorbed by glucose molecules and Φ_{R} is not, the signal ratio of Φ_{S}/Φ_{R} is able to measure the concentration of glucose molecules in the medium. On the other hand, the ill effect of scattering on the measurement is cancelled because both Φ_{S} and Φ_{R} essentially encounter the same sample medium though at a slightly different time. Thus by carefully selecting the values of λ_{S} and λ_{R} for the superfast sequential and alternate dual wavelength pulsating signal processing scheme of the current invention, the very significant ill effect of scattering on the measurement accuracy of glucose concentration levels in whole blood and/or interstitial fluid can be significantly suppressed.

As stated earlier, there are three viable absorption lines of glucose in the NIR (^{˜}0.9-1.5μ) spectral region for the current invention. They are respectively 0.960μ (10,400 cm⁻¹), 1.15082 (8,693 cm⁻¹) and 1.409μ (7,100 cm⁻¹). The 1.409μ absorption line is the strongest of the three but the liquid water background attenuation is also the strongest, amounting to almost 10 cm⁻¹. In order to take advantage of the strength of the 1.409μ absorption line of glucose but not the drawback of a high liquid water background attenuation, a reflection sampling method is used in the current invention as versus using a direct infrared absorption arrangement. This reflection sampling method is now described in more detail below.

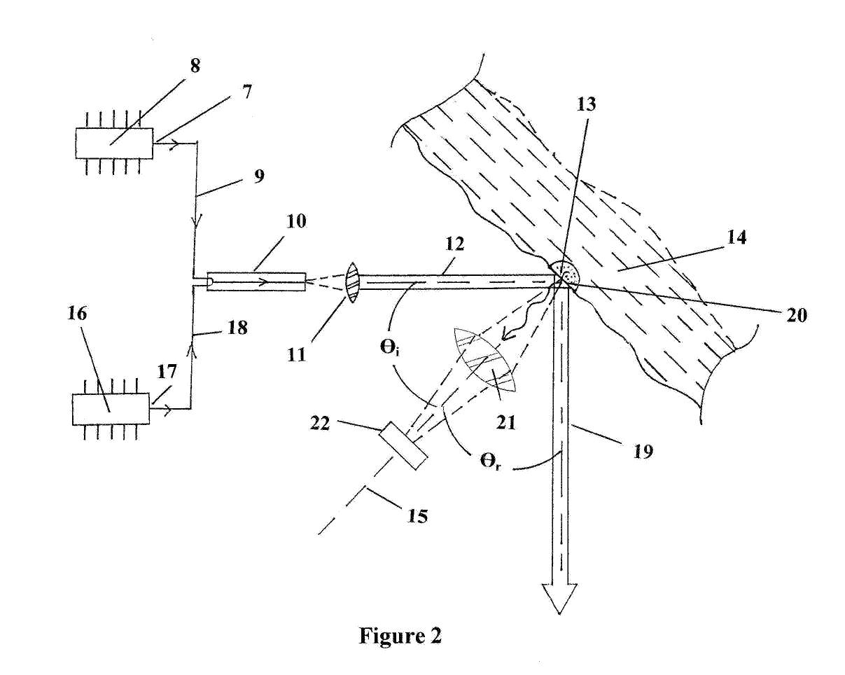

FIG. 2 depicts schematically a specially designed optical system using a reflection sampling technique that can be used to suppress the ill effects of radiation scattering in the ratio output of Φ_{S}/Φ_{R} (S=V_{S}/V_{R}) due to the presence of constituent molecules, including those of glucose and liquid water, in the sampling matrix of whole blood and/or interstitial fluid. This optical system is specially designed to allow two laser diodes of different emitting output wavelengths λ_{S} and λ_{R} (the Φ_{S} and Φ_{R} beams) to be pulsed sequentially and alternately at a superfast rate (^{˜}10-100 KHz) with ^{˜}10% duty factor. Both Φ_{S} and Φ_{R} are directed at the same spot of the sampling matrix arriving alternately and sequentially in time separated only by a few μs. Due to the fact that the molecules within the sampling matrix move very slowly (^{˜}several mm/sec), both the Φ_{S} and Φ_{S} beams literally traverse the same sample medium even though at slightly different times as if the medium is effectively unchanged.

As shown in FIG. 2, the output 7 at the 1.409μ pulsed semiconductor laser diode module 8 is coupled into an optical fiber 9. The output of signal source 8 from optical fiber 9 is then coupled into a multiplexed optical fiber 10 the output of which is collimated by lens 11 into a narrow beam 12 having a diameter of ^{˜}1.0 mm before impinging at spot 13 of the sampling area 14 at an inclined angle θᵢ to the normal 15. The output 17 at the 1.298μ pulsed semiconductor laser diode reference source 16 is coupled into an optical fiber 18 and then into the multiplexed optical fiber 10 the output of which is also collimated by lens 11 into a narrow beam 12 having a diameter of ^{˜}1 mm before incident at the same spot 13 of the sampling area 14. Both the signal beam Φ_{S} (1.409μ) and the reference beam Φ_{R} (1.298μ) are also sequentially and alternately specularly reflected at the same spot 13 into beam 19 which is not being used in the current invention. Both the Signal beam Φ_{S} and the Reference beam Φ_{R} penetrate a small distance into the sampling area 14 before emanating from the sampling area surface 20 at spot 13. The radiation coming out from spot 13 is collected by lens 21 onto detector 22 for signal processing.

Due to the fact that both the Signal and the Reference laser diode modules (8 and 16, respectively) are alternately and sequentially pulsed at a superfast rate, both Signal source radiation 7 and Reference source radiation 17 are ‘combined’ via optical fiber multiplexer into a single beam 12 before entering the sampling area 14 at the same spot 13. After interacting with constituent molecules a short distance inside the sampling area 14 both beams come out and are collected by lens 21 onto detector 22 for signal processing. Due to the superfast sequential and alternate pulsation rate of the two laser diodes and the closeness of their emission wavelengths, the absorptive loss of the intensity because of scattering for both Signal and Reference beams while traversing the short distance inside the sampling area 14 as measured by the detector 22 is almost identical. Therefore if the overall signal of the sensor system is processed as the ratio of the intensities of the two beams as measured by the detector 22, the effect of the absorptive loss or attenuation due to scattering by the molecules of the sampling matrix 14 within the short distance traversed, but not by the absorption of glucose molecules, is almost completely suppressed. But since only the intensity of Φ_{S} is affected by glucose molecules because of specific absorption, the value of the processed ratio, namely S=Φ_{S}/Φ_{R}=V_{S}/V_{R} will reflect the concentration of glucose molecules present in the matrix medium.

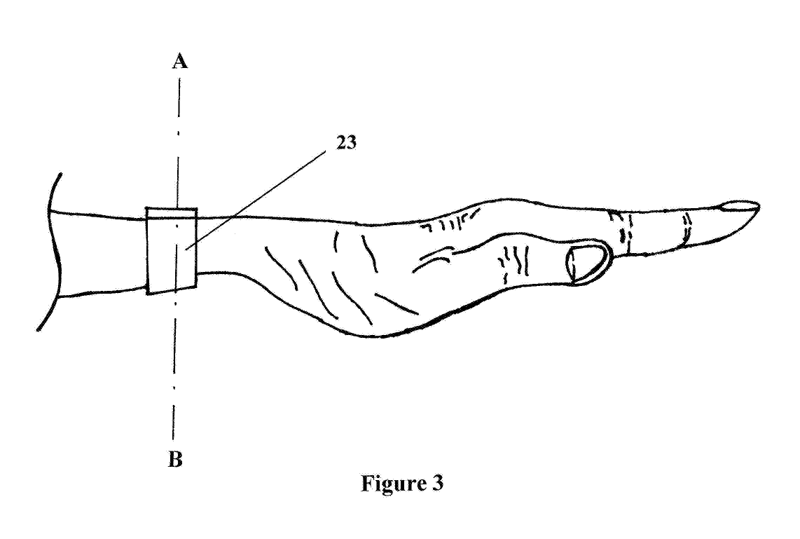

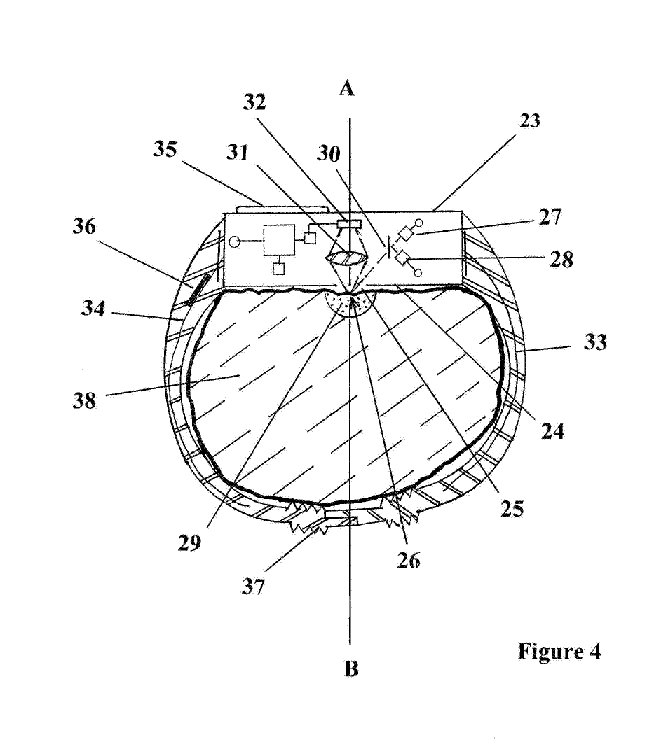

With reference to FIG. 2, sample area 14 represents a body part of a human patient which is used for determining the glucose concentration level in the subject's blood and/or interstitial fluid. The reflection sampling technique disclosed in the present invention makes possible many candidate sampling sites for such a sensor. Just about any exposed human skin surface (epidermis) can be qualified as one. As it turns out, an especially preferred sampling site is the skin surface on the top part of one's wrist where a watch is normally worn. One of the reasons why such a site is especially preferred is that this sampling area can easily accommodate a watch-like design for the sensor such as that portrayed in FIG. 3 where sensor 23 is shown. The cross-sectional view as indicated by the dissecting line AB in FIG. 3 is depicted in FIG. 4. With reference to FIG. 4, bottom 24 of sensor 23 has an opening 25 to the skin surface of wrist 38 at which probing radiations impinge at point 26. The impinging radiations are Signal beam Φ_{S} and Reference beam Φ_{R} generated, respectively, by signal laser module 27 and reference laser module 28. As described earlier, the wavelength of Φ_{S} coincides with one of the absorption lines of glucose molecules, e.g. 1.409μ, and the wavelength of Φ_{R} does not coincides with any absorption lines of glucose molecules, e.g. 1.278μ. Additionally, both Φ_{S} and Φ_{R} are alternately and sequentially pulsed at a superfast rate. They are also combined into a single beam with the use of multiplexer 30 before impinging at the same spot 26. After interacting with constituent molecules at a short distance inside the sampling area 29, both beams come out and are collected by lens 31 onto detector 32 for signal processing.

The ultra-low-noise current driver circuits for the laser modules 27 and 28 are fabricated and housed in flexible printed circuit 33. Flexible circuit 33 also houses the power supply battery for the sensor (not shown in FIG. 4). The low-noise amplifier and processing circuit for the detector 32 is fabricated and housed in flexible circuit 34. Flexible circuit 34 also houses the circuit for driving the LED display 35 which shows the instantaneous blood glucose concentration measured for the person under test. Flexible circuit 34 also includes a Wi-Fi transmitting module 36 which allows the sensor to communicate with the Internet through one or more local network access stations (hotspots) available in the vicinity of the sensor. The clamping mechanism 37 for the watch-like non-invasive blood glucose sensor is also depicted in FIG. 4.

While the invention has been described herein with reference to a preferred embodiment, this embodiment has been presented by way of example only, and not to limit the scope of the invention. Additional embodiments thereof will be obvious to those skilled in the art having the benefit of this detailed description. Further modifications are also possible in alternate embodiments without departing from the inventive concept.

What is claimed is:

1. A method for non-invasively measuring a glucose concentration in an in-vivo sample through use of a non-invasive monitor, comprising:
combining a signal beam Φ_{S} and a reference beam Φ_{R} in a multiplex fashion so that the beams are sequentially and alternately pulsed at a cycle speed toward an impinging site of a surface of the in-vivo sample of a subject with an incident direction subtending an incident angle from the surface normal at the impinging site;
collecting onto a detector both an emerging signal beam Φ_{ES} and an emerging reference beam Φ_{ER} that emerge from the in-vivo sample at the surface using a focusing lens with a collecting angle less than the incident angle;
measuring a value of a ratio of Φ_{ES}/Φ_{ER} at the detector; and
using the value of the ratio of Φ_{ES}/Φ_{ER} to determine the glucose concentration of the subject;
wherein the signal beam Φ_{S} has an absorption peak at CWL=λ_{S};
wherein λ_{S} is an absorption line of glucose having a liquid water attenuation no greater than ^{˜}1.0 cm⁻¹;
wherein the reference beam Φ_{R} has an absorption peak CWL=λ_{R};
wherein λ_{R} has no or negligible absorption by glucose molecules; and
wherein the cycle speed is sufficiently fast so that motion of constituent molecules surrounding glucose in the in-vivo sample are effectively stationary for purposes of determining the glucose concentration of the subject.

2. The method of claim 1 wherein the cycle speed is 10 Khz or faster.

3. The method of claim 3 wherein λ_{S}=1.409μ with FWHM=+/−0.0054μ and λ_{R}=1.28μ with FWHM=+/−0.0054μ.

4. The method of claim 3 wherein λ_{S}=1.150μ with FWHM=+/−0.0054μ.

5. The method of claim 3 wherein λ_{S}=0.960μ with FWHM=+/−0.0054μ.

6. The method of claim 1 wherein attenuation of both Φ_{S} and Φ_{R} by virtue of scattering outside of the incident direction is substantially the same because λ_{S}≈λ_{R}.

7. An apparatus for non-invasively measuring a glucose concentration in an in-vivo sample, comprising:
a signal source semiconductor laser diode which emits a signal beam Φ_{S} having an absorption peak at CWL=λ_{S};
a reference source semiconductor laser diode which emits a reference beam Φ_{R} having an absorption peak at CWL=λ_{R};
multiplex combination means for combining outputs of the signal source and the reference source in a multiplex fashion at a cycle speed such that both beams traverse sequentially and alternately at different times a same distance in and out of an in-vivo sample of a subject after impinging an incident spot of a surface of the in-vivo sample at an incident direction subtending an incident angle from the surface normal at the impinging site;
a detector;
a focusing lens which collects onto the detector both an emerging signal beam Φ_{ES} and an emerging reference beam Φ_{ER} that emerge from the in-vivo sample at the surface; and
electronics for measuring a value of a ratio of Φ_{ES}/Φ_{ER} at the detector and using the value of the ratio of Φ_{ES}/Φ_{ER} to determine the glucose concentration of the subject;
wherein λ_{S} is an absorption line of glucose having a liquid water attenuation no greater than ^{˜}1.0 cm⁻¹;
wherein λ_{R} has no or negligible absorption by glucose molecules; and
wherein the cycle speed is sufficiently fast so that motion of constituent molecules surrounding glucose in the in-vivo sample are effectively stationary for purposes of determining the glucose concentration of the subject.

8. The apparatus of claim 7 wherein the focusing lens has a collecting angle less than an incident angle.

9. The apparatus of claim 7 wherein the cycle speed is 10 Khz or faster.

10. The apparatus of claim 7 wherein λ_{S}=1.409μ with FWHM=0.0054μ and λ_{R}=1.28μ with FWHM=0.0054μ.

11. The apparatus of claim 7 wherein λ_{S}=1.150μ with FWHM=0.0054μ.

12. The apparatus of claim 7 wherein λ_{S}=0.960μ with FWHM=0.0054μ.

13. The apparatus of claim 7 wherein attenuation of both Φ_{S} and Φ_{R} by virtue of scattering outside of the incident direction is substantially the same because λ_{S}≈λ_{R}.

14. The apparatus of claim 7 further comprising a band for wearing the apparatus on a wrist of the subject.

15. The apparatus of claim 14 further comprising electronics for sending the glucose concentration of the subject to a computer.

16. The apparatus of claim 15 wherein the computer is comprised of a smart phone.
